(19) United States
(12) Patent Application Publication
(10) Pub. No.: US 2021/0394762 A1
(43) Pub. Date: Dec. 23, 2021

(54) SYSTEM INTERACTING WITH AN OCCUPANT OF A MOTOR VEHICLE

(71) Applicant: Valeo Systemes Thermiques, Le Mesnil-Saint-Denis (FR)

(72) Inventors: Daniel Neveu, Le Mesnil Saint-Denis Cedex (FR); Georges De Pelsemaeker, Le Mesnil Saint Denis Cedex (FR); Antoine Boilevin, Le Mesnil Saint Denis (FR); Mateo Lopez, Le Mesnil Saint-Denis Cedex (FR)

(73) Assignee: Valeo Systemes Thermiques, Le Mesnil-Saint-Denis (FR)

(21) Appl. No.: 17/295,251

(22) PCT Filed: Nov. 15, 2019
(86) PCT No.: PCT/FR2019/052722

§ 371 Date: May 19, 2021

(30) Foreign Application Priority Data
Nov. 21, 2018 (FR) 1871681

(51) Int. Cl. B60W 40/08; B60W 10/30; B60W 50/00; A61M 21/00; G16H 50/20; G16H 20/70

(52) U.S. Cl. CPC B60W 40/08; A61M 2021/0022; B60W 50/00; A61M 21/00; G16H 50/20; G16H 20/70; B60W 2540/22; B60W 2420/40; B60W 2420/52; B60W 2050/0029; A61M 2205/3313; A61M 2205/3317; A61M 2021/0016; A61M 2021/0027; A61M 2021/0044; B60W 10/30

(57) ABSTRACT

The invention relates to a system that interacts with an occupant of a motor vehicle, comprising: —a measuring device comprising at least one sensor arranged to capture at least one parameter relating to the occupant of said vehicle, —an on-board processing unit using an evaluation model for the emotional state of the occupant, said processing unit being arranged to receive said parameter and to define a data item representative of the emotional state of said occupant using the model, —the representative data corresponding to a point in a three-dimensional space for characterising the emotional state of the occupant, and —at least one actuator configured to activate at least one multi-sensory stimulus for interaction with the occupant, said stimulus allowing the emotional state of said occupant to be altered.

The invention relates to an interactive system which interacts with an occupant of a motor vehicle. The invention also relates to a method for interacting with an occupant of a motor vehicle.

The transition to autonomous automobiles is presenting more and more challenges with regard to creating an empathetic vehicle which will allow driving and/or the journey to be made more pleasant. The progress still to be made may be categorized into three areas encompassing safety, comfort and entertainment.

Some examples of existing systems allow the emotional state of an occupant of a motor vehicle to be evaluated. Specifically, it is known practice to carry out measurements allowing various parameters relating to an occupant to be determined, and thus a datum representative of his emotional state to be derived therefrom.

In general, the measurements may be carried out with the aid of wearable devices allowing data such as the conductivity of the skin, temperature, or indeed heart rhythm to be collected. The system used comprises, if possible, a camera, for example of GoPro® type.

In the known systems, the parameters obtained are then used to determine the emotional state of the occupant. A known model consists in obtaining, on the basis of the various parameters, a mapping of the emotional state of the passenger. Thus, the latter may be represented by a point in a two-dimensional space formed of an x-axis corresponding to valence (intrinsically pleasant or unpleasant quality of a stimulus or of a situation) and a y-axis corresponding to arousal (strength of the emotional stimulus).

The invention aims to improve the known systems by providing an interactive system allowing the emotional state of an occupant of a vehicle to be modeled, not only by placing it in a three-dimensional space, but also by interpreting it so as to integrate supplementary parameters into the model used. Moreover, the invention also proposes to act on the occupant depending on the emotional state detected and thus to have an impact on the safety and/or the comfort of the one or more occupants of the vehicle.

One subject of the invention is an interactive system which interacts with an occupant of a motor vehicle, comprising:
(a) a measurement device comprising at least one detector arranged to acquire at least one parameter connected with the occupant of said vehicle,
(b) a built-in processing unit using a model for evaluating the emotional state of the occupant, said processing unit being arranged to receive said parameter and to define a datum representative of the emotional state of said occupant by means of said model,
(c) the representative datum corresponding to a point in a three-dimensional space for characterizing the emotional state of the occupant,
(d) at least one actuator configured to activate at least one multisensory stimulus in order to interact with the occupant, said stimulus allowing the emotional state of said occupant to be modified.

According to one aspect of the invention, the detectors are composed of at least an ultra-wideband radar and of an infrared camera.

According to one aspect of the invention, the ultra-wideband radar has a frequency between 10 GHz and 1 THz, in particular between 50 GHz and 160 GHz.

According to one aspect of the invention, the infrared camera detects wavelengths between 0.7 μm and 100 μm, in particular between 25 μm and 100 μm.

According to one aspect of the invention, the three-dimensional space for characterizing the emotional state is formed of a first axis corresponding to valence, of a second axis corresponding to dominance and of a third axis corresponding to arousal.

According to one aspect of the invention, the representative datum is preprocessed before activating the at least one multisensory stimulus.

According to one aspect of the invention, the representative datum placed in the 3D space will be correlated with one or more mappings corresponding to various stimuli.

According to one aspect of the invention, the preprocessing consists in taking account of supplementary data, in particular the culture and/or the education of the occupant and/or the personal experience and life of said occupant.

According to one aspect of the invention, the supplementary data may be determined by means of an autonomous measurement system allowing air quality and/or pollution and/or microparticles and/or allergens and/or the sound environment and/or the light environment and/or temperature and/or humidity level to be measured.

According to one aspect of the invention, the evaluation model is created by means of artificial intelligence.

According to one aspect of the invention, the at least one multisensory stimulus is a perfume and/or essential oils and/or a nebulizer and/or lighting and/or a sound and/or music and/or a vibration and/or a massage and/or an air flow.

According to one aspect of the invention, the multisensory stimulus allows the emotional state of the occupant to be modified.

According to one aspect of the invention, the multisensory stimulus allows the emotional state of the occupant to be remediated.

According to one aspect of the invention, the multisensory stimulus allows the emotional state of the occupant to be amplified.

According to one aspect of the invention, the interactive system has a learning loop allowing the processing of the model for evaluating emotion to be improved.

According to one aspect of the invention, the at least one detector may be chosen from: a camera, in particular a far-infrared and/or near-infrared and/or visible-range camera, a microphone, a wearable device, a detector installed in the vehicle, in particular a conductive element placed in a seat and/or in a steering wheel and/or in an armrest.

According to one aspect of the invention, the interactive system may comprise at least a far-infrared camera, a plurality of detectors of vital signs and a microphone.

According to one aspect of the invention, the interactive system comprises at least one display means, particularly a user interface.

According to one aspect of the invention, the at least one display means may be a wearable device, in particular a watch and/or glasses and/or a bracelet and/or a belt and/or a shirt.

According to one aspect of the invention, the at least one display means may be a text and/or a color and/or a sound and/or a vibration.

The invention also relates to a method for interacting with an occupant of a motor vehicle, comprising the steps consisting in:
a) measuring a parameter connected with an occupant of the vehicle by means of at least one detector,
b) receiving the parameter by means of a processing unit and determining a datum representative of the emotional state of the occupant,
c) analyzing the representative datum and placing it in a three-dimensional space for characterizing the emotional state of the occupant,
d) interpreting the representative datum by adjusting the three-dimensional space depending on supplementary data, in particular the culture and/or the education of the occupant and/or the personal experience and life of said occupant,
e) actuating an actuator so as to activate at least one multisensory stimulus in order to interact with the occupant, said stimulus allowing the emotional state of said occupant of the interior of the vehicle to be modified in order to interact with the occupant depending on said datum.

According to one aspect of the invention, the method furthermore comprises a step consisting in informing the occupant of his emotional state and/or of the stimulus applied.

The invention will be better understood and other details, features and advantages of the invention will become apparent on reading the following description, which is given by way of non-limiting example with reference to the appended drawing, in which:

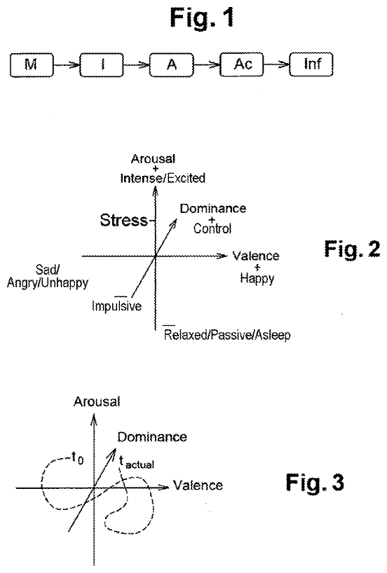

FIG. 1 is a schematic diagram of at least a part of the method according to the invention,

FIG. 2 is a schematic representation of a three-dimensional space AVD used in one of the steps of the method of FIG. 1,

FIG. 3 is a schematic representation of the evaluation step of the method of FIG. 1,

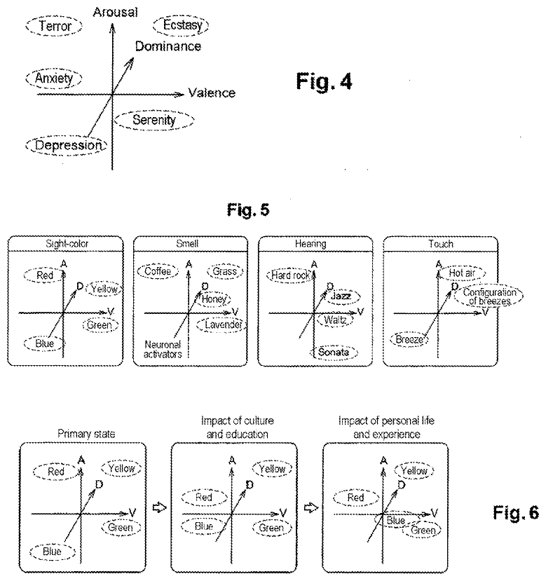

FIG. 4 is a schematic representation of the analysis step of the method of FIG. 1,

FIG. 5 is a schematic representation of the multisensory mapping used in the method of FIG. 1,

FIG. 6 is a schematic representation of the construction of the mapping of FIG. 5,

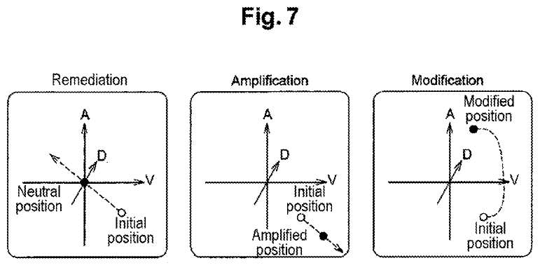

FIG. 7 is a schematic representation of the action step of the method of FIG. 1.

The interactive system proposed in the invention uses a method having five steps. These five steps, which are represented schematically in FIG. 1, consist of:
a) a measurement step (M) carried out by a measurement device and during which one or more parameters relating to the one or more occupants are collected,
b) an evaluation and interpretation step (I) during which the parameters obtained are modeled so as to obtain a datum representative of the emotional state of the one or more occupants,
c) an analysis step (A) making it possible to map the emotional state of the occupant and to decide whether or not an action is necessary,
d) an action step (Ac) during which at least one multisensory stimulus is applied so as to modify the emotional state of the one or more occupants,
e) an information step (Inf) during which the occupant is informed of his emotional state and of the stimuli applied.

During the measurement step (M), the measurement device will acquire one or more parameters describing the status and therefore the emotional state of the occupant (what is described for one occupant may of course be applied to a plurality of occupants of the vehicle simultaneously). Parameters describing the environment in which the occupant finds himself may also be collected so as to derive therefrom their potential effect on the emotional state of the occupant.

The device described in the preceding paragraphs is composed of one or more detectors. These detectors are preferably detectors built into the vehicle, such as for example one or more cameras, detectors of vital signs, one or more microphones or indeed contact detectors.

In one preferred embodiment, the measurements are carried out by cameras, in particular infrared cameras which take images in the infrared domain. These cameras are directed toward the expected positions of the various occupants of the vehicle: the driver's seat, passenger seat, rear bench seat, etc. In particular, one or more very wide-angle cameras (for example of the “fisheye” type) may cover a plurality of positions simultaneously. The infrared cameras preferably detect wavelengths between 0.7 μm and 100 μm, preferably 25 μm and 100 μm.

These cameras comprise, advantageously, near-infrared (NIR) cameras and/or far-infrared (FIR) cameras.

The images from the near-infrared cameras may for example serve to delimit the position, dimensions and movements of various parts of the body of an occupant of the vehicle. The images from the far-infrared cameras may for example serve to identify the parts of the body of the occupant exchanging the most heat with the vehicle interior, for example the head and hands, which are not covered with clothes and will thus appear hotter.

As explained above, the measurement device also allows the environment of the one or more passengers to be determined, the environment possibly influencing their emotional state. Thus, data such as temperature, luminous intensity, noise, the speed of the vehicle, etc. will possibly be collected.

Other types of parameters will also be collected by means of other detectors.

The measurement device comprises at least one microphone allowing the voice of the one or more passengers to be recorded.

The measurement device may also comprise biosensors able to detect parameters such as organic compounds, ions, bacteria, etc.

The measurement device may also be composed of detectors of vital signs. The latter may for example take the form of contactless detectors such as for example a radar, a camera, or indeed wearable elements (watch, shirt, bracelet, etc.).

Sensors of vital signs in contact with the passenger may also be used. They take, for example, the form of conductive elements installed in parts of the vehicle (armrest, steering wheel, seat, etc.). These detectors of vital signs measure parameters such as heart rhythm and/or frequency, breathing frequency and/or amplitude, the conductivity of the skin, brain waves, etc.

One preferred embodiment uses an ultra-wideband short-range radar. The frequency of the radar may for example be between 10 GHz and 1 THz, preferably between 50 GHz and 160 GHz.

The following interpretation and/or evaluation step (I) consists in evaluating and interpreting the parameters obtained during the measurement step (M). Thus, these parameters will be translated into a representative datum easily represented in a first three-dimensional space or 3D space (AVD) for characterizing emotional state. This 3D space is represented in FIG. 3. It is formed of a first axis corresponding to valence (V), of a second axis corresponding to dominance (D) and of a third axis corresponding to arousal (A).

Valence characterizes the level of pleasure or unpleasantness associated with an emotion while arousal may be defined as being the intensity of the affects and of the response generated by this emotion. Dominance, for its part, characterizes the level of mastery and control of the response of the individual to this emotion.

FIG. 3 illustrates the placement of the previously described representative datum obtained with the aid of a processing unit using a model for evaluating emotions, in the three-dimensional space AVD. This datum varies over the course of the time spent by the occupant in the vehicle. Thus, a first representative datum is provided for a time to while the representative datum corresponding to the present is illustrated by t_{actual}. Continuous measurements are preferably carried out, so as to obtain the curve represented in FIG. 3. However, discrete measurements may also be performed.

Once the representative datum has been evaluated, an analysis step (A) is performed. This analysis (A) allows the emotional state of the occupant to be determined depending on the placement of the representative datum in the 3D space AVD.

An example of the previously described mapping is represented in FIG. 4. This mapping of the emotional state of the occupant will then be correlated with one or more mappings corresponding to various stimuli. These stimuli are based on the five senses: sight, smell, taste, touch, hearing. The mappings corresponding to each of the five senses are represented in FIG. 5.

The mapping of the five senses is carried out in three steps, which are illustrated in FIG. 6. Specifically, the initial settings of the vehicle may be described as being those corresponding to the primary emotional state of the occupant, that is to say to an emotional state in which the occupant primarily reacts.

The mapping is then modified to take account of the impact of the culture and/or of the education of the occupant. This step may, for example, be carried out by deep learning, allowing a user profile to be obtained for each occupant.

The last step in the construction of the mapping relates to the impact of the personal life and/or of the experience of the occupant. This step is preferably performed by machine learning, thus allowing the mapping to be adjusted in real time.

The action step (Ac) represented in FIG. 1 corresponds to the application of one or more stimuli. The choice of the stimulus depends on the desired effect. After having placed the datum representative of the occupant on the mapping adjusted in the manner explained in the preceding paragraphs, various types of actions may be selected. The point of intersection of the three axes of the three-dimensional space AVD corresponds to the neutral position of the emotional state of the occupant.

FIG. 7 illustrates three examples of actions which may be applied. Hence, the choices may be:
a) a remediation, for which a stimulus located symmetrically with respect with the neutral position in the 3D space AVD will be applied. In this way, the emotional state of the occupant is brought back to the neutral position represented in FIG. 7;
b) an amplification, for which a stimulus positioned in the opposite direction to the neutral position in the 3D space (with respect to the initial position) is applied;
c) a modification making it possible to move from the initial position (initial status of the emotional state) to a given specific position (desired status of the emotional state), the one or more stimuli being chosen depending on the given specific position.

The multisensory stimuli used may, for example, be:
a) For sight: use of interior lighting with various colors and/or of various intensities,
b) For smell: use of a diffuser of perfume, essential oils or indeed pheromones,
c) For touch: use of radiant panels, nebulization of water, use of jets of hot and/or cold air,
d) For hearing: selection of ambient noises or music,
e) For taste: diffusion of perfume or nebulization possibly creating pheromones combining the five main flavors (savory, sour, bitter, sweet, umami).

There may be display means allowing the occupant to be informed of his emotional state. The interactive system may have one or more display means. The latter may, for example, take the form of a wearable device, in particular a watch and/or glasses and/or a bracelet and/or a belt and/or a shirt.

The display means may also take the form of a text and/or a color and/or a sound and/or a vibration.

The invention is not limited to the embodiments described in this patent application. Various modifications may be made without departing from the scope of the invention. In particular:
a) the type of stimulus may be different from those described in the patent application,
b) the preprocessing may take account of only some of the five senses to carry out the previously described mapping,
c) the measurements may be carried out simultaneously or one after another for the various occupants of the vehicle,
d) the order of the steps of the method may vary, for example a stimulus may be applied before the step of measuring the parameters.

1. An interactive system which interacts with an occupant of a motor vehicle, comprising:
a measurement device comprising at least one detector arranged to acquire at least one parameter connected with the occupant of said vehicle;
a built-in processing unit using a model for evaluating an emotional state of the occupant, said processing unit being arranged to receive said parameter and to define a datum representative of the emotional state of said occupant by means of said model,
the representative datum corresponding to a point in a three-dimensional space for characterizing the emotional state of the occupant; and
at least one actuator configured to activate at least one multisensory stimulus in order to interact with the occupant, said stimulus allowing the emotional state of said occupant to be modified.

2. The interactive system as claimed in claim 1, wherein the detectors are composed of at least an ultra-wideband radar and of an infrared camera.

3. The interactive system as claimed in claim 2, wherein the ultra-wideband radar has a frequency between 50 GHz and 160 GHz.

4. The interactive system as claimed in claim 2, wherein the infrared camera detects wavelengths between 25 μm and 100 μm.

5. The interactive system as claimed in claim 1, wherein the three-dimensional space for characterizing the emotional state is formed of a first axis corresponding to valence, of a second axis corresponding to dominance and of a third axis corresponding to arousal.

6. The interactive system as claimed in claim 1, wherein the representative datum is preprocessed before activating the at least one multisensory stimulus.

7. The interactive system as claimed in claim 6, wherein the representative datum placed in the 3D space will be correlated with one or more mappings corresponding to various stimuli.

8. The interactive system as claimed in claim 6, wherein the preprocessing consists of taking account of supplementary data comprising culture and/or education of the occupant and/or personal experience and life of said occupant, wherein the supplementary data are added to data corresponding to the primary emotional state of the occupant.

9. The interactive system as claimed in claim 1, wherein the evaluation model is created by means of artificial intelligence.

10. The interactive system as claimed in claim 1, wherein the at least one multisensory stimulus is a perfume and/or essential oils and/or a nebulizer and/or lighting and/or a sound and/or music and/or a vibration and/or a massage and/or an air flow and/or light.

11. The interactive system as claimed in claim 1, wherein the multisensory stimulus is arranged to allow the emotional state of the occupant to be modified.

12. The interactive system as claimed in claim 11, wherein the multisensory stimulus is arranged to allow the emotional state of the occupant to be remediated.

13. The interactive system as claimed in claim 11, wherein the multisensory stimulus is arranged to allow the emotional state of the occupant to be amplified.

14. The interactive system as claimed in claim 1, comprising a learning loop allowing the processing of the model for evaluating emotion to be improved.

15. The interactive system as claimed in claim 1, further comprising at least one display means having a user interface.

16. A method for interacting with an occupant of a motor vehicle, comprising:
measuring a parameter connected with an occupant of the vehicle by at least one detector,
receiving the parameter by a processing unit and determining a datum representative of the emotional state of the occupant,
analyzing the representative datum and placing it in a three-dimensional space for characterizing the emotional state of the occupant,
interpreting the representative datum by adjusting the three-dimensional space depending on supplementary data, the culture and/or the education of the occupant and/or the personal experience and life of said occupant,
actuating an actuator so as to activate at least one multisensory stimulus in order to interact with the occupant, said stimulus being arranged to allow the emotional state of said occupant of the interior of the vehicle to be modified in order to interact with the occupant depending on said datum.

17. The method as claimed in claim 16, further comprising: informing the occupant of his emotional state and/or of the stimulus applied.
